(19) United States
(12) Patent Application Publication
(10) Pub. No.: US 2006/0196502 A1
(43) Pub. Date: Sep. 7, 2006

(54) Oxygen deprivation system

(76) Inventors: Murray Pilcher, Auckland (NZ); Andrew Chapman, Auckland (NZ); Nigel Thomson, Auckland (NZ)

Correspondence Address: JACOBSON HOLMAN PLLC, 400 SEVENTH STREET N.W., SUITE 600, WASHINGTON, DC, 20004, US

(21) Appl. No.: 10/545,686

(22) PCT Filed: Feb. 13, 2004
(86) PCT No.: PCT/NZ04/00027

§ 371 Date: Apr. 27, 2006

(30) Foreign Application Priority Data
Feb. 13, 2003 (NZ) 524183
Feb. 14, 2003 (NZ) 524210
Oct. 10, 2003 (NZ) 528862

(51) Int. Cl. A62B 7/00; A61K 33/00

(52) U.S. Cl. 128/200.24; 424/600

(57) ABSTRACT

A simulated altitude training system for providing deleted oxygen gas at variable levels for a range of users each user has a predetermined plan and has an individual identifier. The system automatically delivers the correct oxygen level according to the prescribed individual plan. Also provided are alternative embodiments for providing the individualised oxygen level as well as a membrane heating system.

### FIELD OF THE INVENTION

The present invention relates to hypoxic treatment particularly though not solely to an automated interval hypoxic training for living organisms.

### BACKGROUND

Simulated Altitude Training (‘SAT’) is the effective delivery of hypoxic (oxygen-reduced) air to the user, which simulates the oxygen content of air at altitude.

Early approaches to altitude training meant travelling to and living at high altitude for extended periods. Training at high altitude forced athletes to decrease their intensity of training as the lack of oxygen while under intense training caused fatigue and prolonged the athletes ability to return to quality performances when back at sea level.

The ‘live high, train low’ model of altitude training was subsequently introduced as it allowed athletes to sleep and live at high levels of altitude and then travel to lower levels to train, meaning the training intensity levels could be maintained. This was an expensive and inconvenient practice that has been demonstrated to provide fewer benefits than more recent technology.

The major technological advance in altitude training was the adoption of altitude simulation developed by the Russian aerospace medicine programme. In essence, this brought the mountain to the athlete.

Altitude simulation now makes it possible to get the benefits of altitude exposure without the expense of travel and time commitments.
SAT is a cost effective alternative to altitude training.
SAT is a natural method of optimising aerobic capacity.
SAT improves the uptake, delivery and utilisation of oxygen.

Altitude simulation was initially achieved with early technologies such as hypobaric (vacuum) sleeping tents and hypoxic living and training chambers. These technologies were cumbersome and impractical and have been surpassed a semi-portable breathing apparatus called a hypoxicator that enables the athlete to alternate the intake of hypoxic (from the hypoxicator) and normal (ambient) air.

Prior Art hypoxicators' extract oxygen from the air to achieve oxygen levels as low as 9% (equivalent to 22,000 feet altitude) which is less than half the oxygen content at sea level (20.9%). An example of the prior art is described in International Patent Publication no. WO96/37176. A system is disclosed which oxygen depleted air is generated and delivered to a mask. The mask is then worn by the subject for the predetermined period and then removed.

### SUMMARY OF THE INVENTION

It is therefore an object of the present invention to overcome any disadvantages in the prior art or to at least provide the public with a useful choice.

In a first aspect the present invention may be broadly said to consist in a system for modifying the composition of air and delivering said modified gas to at least one recipient(s) comprising:

at least one source of gas configured to vary said composition and deliver said modified gas to said recipient(s) depending on a control signal;

at least one sensor configured to determine at least one aspect of said composition; and

at least one controller configured to receive said aspect and provide said control signal to said source based on said aspect, a predetermined desired level or range corresponding to said composition and at least one historical value of said aspect.

In a second aspect the present invention may be broadly said to consist in a system for modifying the composition of air and delivering said modified gas to at least one recipient(s) comprising: at least one source of gas configured to vary said composition and deliver said modified gas to said recipient(s) depending on a control signal;

at least one controller configured to receive identification of said recipient(s) provide said control signal to said source based the progress through a predetermined oxygen profile stored in relation to said recipient(s).

In a third aspect the present invention may be broadly said to consist in a system for modifying the composition of air and delivering said modified gas to at least one recipient(s) comprising:

at least one source of gas configured to vary said composition and deliver said modified gas to said recipient(s) depending on a control signal;

at least one sensor configured to determine at least one aspect of said composition; and

at least one controller configured to receive said aspect and provide said control signal to said source based on a predetermined desired range for said aspectstored in relation to said recipient(s).

Preferably said aspect corresponds to the oxygen level.

Preferably said sensor comprises a non-invasive sensor configured to determine the level of oxygen in the blood of said recipient(s).

Preferably said sensor further comprises at least one sensor to determine the level of oxygen in the gas delivered to said recipient(s).

Preferably said system is configured to deliver gas to two or more recipient(s) completely independently.

Preferably said source further comprises at least two inputs; a first input configured to receive normoxic (normal oxygen level) gas or ambient air, and a second input configured to receive hypoxic (oxygen reduced, depleted or bereft) gas.

Preferably said source further comprises at least one mixer(s) configured to vary the proportion of gas from said first input and said second input according to said control signal.

Preferably said source further comprises at least two said mixers, at one mixer(s) configured to supply each said recipient(s).

Preferably said controller configured to receive the heart rate of said recipient(s) and provide said control signal to said source also based on said heart rate.

Preferably said controller configured to receive the breathing rate of said recipient(s) and provide said control signal to said source also based on said breathing rate.

In a fourth aspect the present invention may be broadly said to consist in a system for modifying the composition of air and delivering said modified gas to at least one recipient(s) comprising:

means for varying said composition and delivering said modified gas to said recipient(s) depending on a control signal;

means for determining at least one aspect of said composition; and

means for receiving said aspect and providing said control signal to said source based on said aspect, a predetermined desired level or range corresponding to said composition and at least one historical value of said aspect.

In a fifth aspect the present invention may be broadly said to consist in a system for modifying the composition of air and delivering said modified gas to at least one recipient(s) comprising:

means for varying said composition and delivering said modified gas to said recipient(s) depending on a control signal;

means for receiving identification of said recipient(s) providing said control signal based progress through a predetermined oxygen profile stored in relation to said recipient(s).

In a sixth aspect the present invention may be broadly said to consist in a system for modifying the composition of air and delivering said modified gas to at least one recipient(s) comprising:

means for varying said composition and delivering said modified gas to said recipient(s) depending on a control signal;

means for determining at least one aspect of said composition; and

means for receiving said aspect and providing said control signal to said source based a predetermined desired range for said aspect.

In a seventh aspect the present invention may be broadly said to consist in a method for modifying the composition of air and delivering said modified gas to at least one recipient(s) comprising:

varying said composition and delivering said modified gas to said recipient(s) depending on a control signal;

determining at least one aspect of said composition; and

receiving said aspect and providing said control signal to said source based on said aspect, a predetermined desired level or range corresponding to said composition and at least one historical value of said aspect.

In a eighth aspect the present invention may be broadly said to consist in a method for modifying the composition of air and delivering said modified gas to at least one recipient(s) comprising:

varying said composition and delivering said modified gas to said recipient(s) depending on a control signal;

receiving identification of said recipient(s) providing said control signal to said source based the progress through a predetermined oxygen profile stored in relation to said recipient(s).

In a ninth aspect the present invention may be broadly said to consist in a method for modifying the composition of air and delivering said modified gas to at least one recipient(s) comprising:

varying said composition and delivering said modified gas to said recipient(s) depending on a control signal;

determining at least one aspect of said composition; and

receiving said aspect and providing said control signal to said source based a predetermined desired range for said aspect.

In a tenth aspect the present invention may be broadly said to consist in a system for modifying the composition of air and delivering said modified gas to at least one recipient(s) comprising:

at least one oxygen reducer reducing the oxygen component of said gas;

at least one heater adapted to heat said gas before it enters said reducer and/or said reducer directly.

Preferably said heater is configured to provide a first level of heating at start up and a second operating level of heating.

Preferably said heater is configured to maintain at least a component of said reducer within a predetermined range.

Preferably said range is 40-45° C.

In an eleventh aspect the present invention may be broadly said to consist in a system for modifying the composition of air and delivering said modified gas to at least one recipient(s) comprising:

means for reducing the oxygen component of said gas;

means for heating said gas before it enters said reducing means and/or said reducing means directly.

In a twelfth aspect the present invention may be broadly said to consist in a method of modifying the composition of air and delivering said modified gas to at least one recipient(s) comprising:

reducing the oxygen component of said gas;

heating said gas before reduction and/or said heating reduction apparatus directly.

In a thirteenth aspect the present invention may be broadly said to consist in a system for modifying the composition of air and delivering said modified gas to at least one recipient(s) comprising:

at least one source of low oxygen gas at above ambient pressure;

at least one source of ambient air;

at least one mixer or venturi including a gas inlet configured to connect to said low oxygen source, a variable throat inlet configured to connect to said low said ambient air source, and at least one controller configured to vary said throat inlet to achieve a predetermined proportion or range of oxygen at the gas output from said mixer.

Preferably said oxygen sensor providing an indication of the oxygen level at said gas outlet, and said controller configured to vary said throat inlet at least based on said predetermined proportion and said indication of said oxygen level.

Preferably said oxygen sensor further comprising a receptacle to receive scented material proximate or integrated with said throat inlet.

In a fourteenth aspect the present invention may be broadly said to consist in a system for modifying the composition of air and delivering said modified gas to at least one recipient(s) comprising:

means for providing low oxygen gas at above ambient pressure;

means for providing ambient air;

means for combining said low oxygen gas with said ambient air to achieve a predetermined proportion of oxygen in the gas output from said mixer.

In a fifteenth aspect the present invention may be broadly said to consist in a method for modifying the composition of air and delivering said modified gas to at least one recipient(s) comprising:

providing low oxygen gas at above ambient pressure;

providing ambient air;

combining said low oxygen gas with said ambient air using a venturi to achieve a predetermined proportion of oxygen in the gas output from said mixer.

In a sixteenth aspect the present invention may be broadly said to consist in a system for modifying the composition of air and delivering said modified gas to a recipient as described as any of the embodiments herein and as illustrated by any of the accompanying drawings.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The invention consists in the foregoing and also envisages construction which the following gives examples.

### BRIEF DESCRIPTION OF THE DRAWINGS

One preferred form of the present invention will now be described with reference to the accompanying drawings in which

FIG. 1 is block diagram of the present invention in use with a single user;

FIG. 2 is a pneumatic diagram of the gas supply;

FIG. 3 is a schematic diagram of the supply controller;

FIG. 4 is a wiring diagram of the gas supply;

FIG. 5 is a pneumatic diagram of the delivery apparatus;

FIG. 6 is a wiring diagram of the delivery apparatus;

FIG. 7 is a schematic diagram of the delivery controller;

FIG. 8 is a flow diagram depicting the operating procedure;

FIG. 9 is a schematic diagram of the client interface;

FIG. 10 is a section showing the venturi controlling the O₂ level;

FIG. 11 is a section showing the operation of the heat exchanger;

FIG. 12 is a pneumatic diagram of a further embodiment with the heat exchange and venturi;

FIG. 13 is a pneumatic diagram of a further embodiment with multiple outlets.

### DETAILED DESCRIPTION

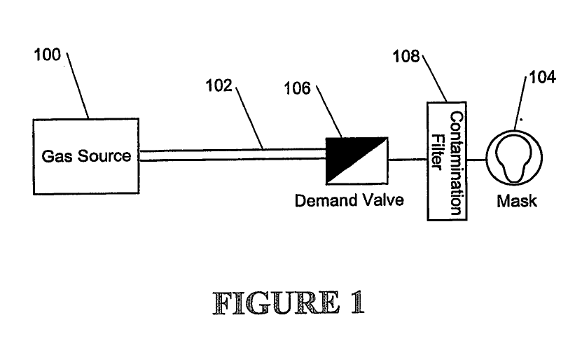

Referring to FIG. 1 the present invention is depicted in one embodiment with a gas source 100 connected through a conduit 102 to a medical resuscitation demand valve 106 designed to deliver up to 15 l/min. The demand valve delivers the air mixture when the user inhales by a diaphragm or pressure sensor that operates a mechanical or electronic valve that releases the air into a standard resuscitation mask directly or via a cross contamination filter 108. The demand valve takes the air mixture at an input pressure between 2.8 to 3.5 bar and reduces this pressure to a normal atmosphere through the mechanical or electronic valve into the mask.

The mask 104 for example comprises a standard resuscitation mask consisting of a plastic housing that has an attachment nozzle to fit either the demand valve or the cross contamination filter and attachments for a strap or harness that fits the mask to the face/head of the user. An air filled cushion or soft rubber type cushions makes an airtight fit between the user and the mask. The cross contamination filter is a plastic housing containing a replaceable filter media such as a treated paper and attached to the demand valve and mask by tubular nozzles on either side of the filter media delivering gas to a user. The filter media is an anaesthetic bacterial grade viral and bacterial filter. Its purpose is to eliminate the potential of contamination of bacteria and germs being passed from user to user.

While in FIG. 1 the user is depicted as a human, the system and method could equally be employed with any living organism animal or plant. It will be appreciated that in certain embodiments variation of a gas other than oxygen might be appropriate, for example with plants varying CO₂ levels.

In one embodiment the gas delivered to the user is varied in oxygen content according to a predetermined profile specifically designed for that user. The present invention keeps track of the user's progress and varies the treatment over the profile depending on the type of treatment required.

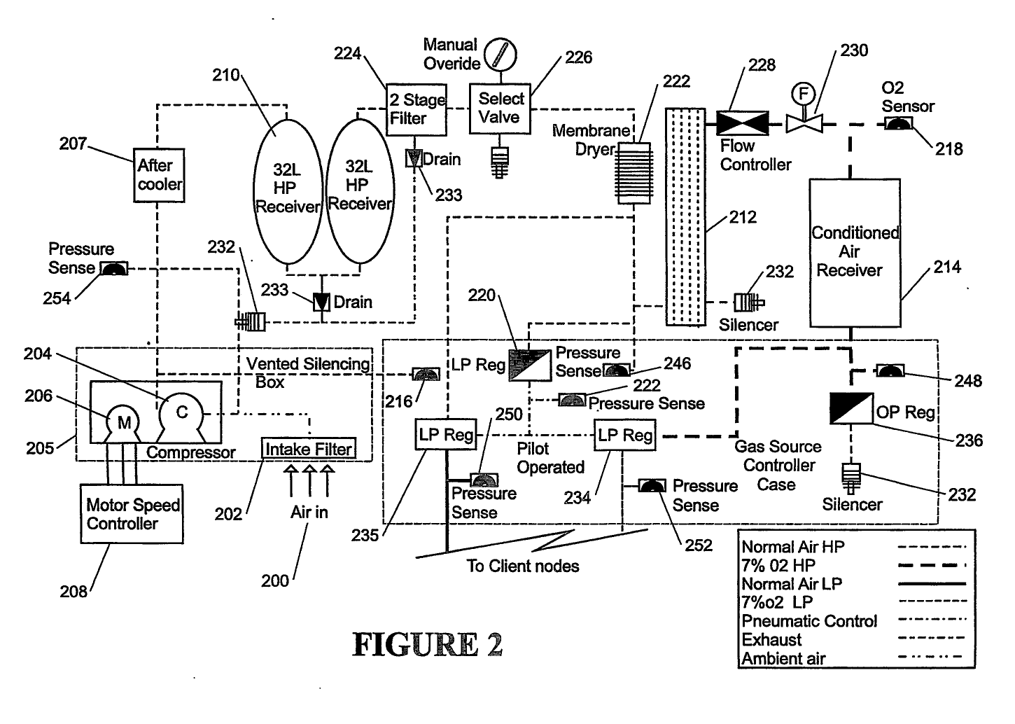

Referring to FIG. 2 we see one embodiment of the gas source 100 in more detail. Air inlet 200 includes an intake filter 202 consisting of a replaceable paper media inside a plastic and metal housing the filter removes particles above 5 microns in size, supplying atmospheric pressure to the low pressure inlet of compressor 204. The compressor 204 preferably comprises an oil free scroll compressor delivering 240 standard litres a minute of compressed air at 8 bar±1 bar. The compressor is preferably driven by electric motor 206 2.2 kw single phase motor. Electric motor 206 is speed controlled by Motor Speed Controller 208

Normal air receiver 210 is preferably a 2×16 litre steel pressure vessel, which receives the High Pressure (HP) normal air from the compressor 204 and store it at 8±1 bar. This provides a buffer of pressurised air to cope with instantaneously changes in system load. The motor speed controller 208 provides closed loop control of the pressure in the main receiving vessels 210 by sensing the pressure in the vessels via sensor 216.

In doing so with speed control the lifetime of the compressor motor can be extended by reducing the fully powered stop start cycle. The buffering avoids continual off/on operation triggered by any drop in the pressure of the air stored in the normal air receivers 210 the conditioned air receiver 214 and the nitrogen generator 212. The pressure fluctuations are created by the differing air consumption of the individual users and the number of users connected to the machine and the variations in the treatment programmes of the individual users. The Motor Speed controller also allows for more efficient sizing of the conditioned air and normal air receivers.

The motor 206 and compressor 204 are contained in a vented silencing box 205 that uses an acoustic damping material and a variety of baffles to reduce the transmitted noise of the compressor and motor while still allowing for adequate airflow for cooling both compressor and motor.

The outer housing uses similar acoustic damping materials and baffles to reduce the noise of the air intake of the compressor and the cooling air intake for the motor and compressor and the heated exhaust air from the same as well as reducing the noise from the various silencer devices.

A two stage filter 224 removes particles and moisture with a coalescing media and odour using activated charcoal media, from the HP normal air from the receivers 210.

Main shut off valve 226 turns off the air supply from the normal air receivers 210 and is a solenoid operated three port valve.

An air dryer 222 after the valve 226 reduces the moisture content of the air either by temperature control or by a coalescing filter media.

A Nitrogen generator 212 uses a specialised filter to remove oxygen from air. The amount of oxygen removed is controlled by air intake pressure airflow and air temperature. In the preferred embodiment a nitrogen purifier membrane is used. Removed oxygen is exhausted from the membrane via a silencer 232.

Flow control valve 228 is a solenoid operated variable output valve used as a pressure regulator and to vary the composition of the delivered gas mixture by restricting the output flow of the nitrogen generator 212, as measure by flow sensor 230.

The receivers and filters preferably include drain valves 233 to remove any accumulated condensation or moisture. Each drain valve includes one way/non return valves and an electronically controlled valve. The controller 500 opens the valves periodically eg: every 20 min for a short period eg: a couple of seconds to release water. The water is gravity and pressure driven out of the valve.

Conditioned air receiver 214 (1×10 litres) is a steel pressure vessel which stores the conditioned air at 8 bar±1 bar from the regulated output from the nitrogen generator.

A number of pressure sensors measure the pressure in the airline connecting various components. Pressure sensor 216 measures pressure from normal air receivers 210. Pressure sensor 222 measures pressure out of the pilot controlling regulator 220. Pressure sensor 246 measures pressure out of nitrogen generator 212. Pressure sensor 248 measures pressure out of conditioned air receiver 214. Oxygen Sensor 218 measures the oxygen content of the air as it leaves the nitrogen generator 212.

Pilot control regulator 220 is a mechanical or electronic valve to control the pressure of pilot operated regulators (detailed below) via a diaphragm that operates a mechanical valve or a pressure sensor operating a proportional solenoid operated valve.

Pilot operated regulator 234 is a pressure controlling device using a pilot air supply to ensure the same pressure in the conditioned 234 air supply by a diaphragm operated control valve. The normal air supply is regulated by Pilot operated regulator 235.

Over pressure regulator 236 is a proportional solenoid operated valve used in conjunction with a pressure sensor to regulate the pressure of conditioned air from the conditioned air receiver.

Silencers 232 are used to diffuse the air or gas expelled at pressure above normal atmosphere though a baffle media to reduce sound from the normal receiver 210 drain, the filter 224 drain, the nitrogen generator 212 and the OP regulator 236.

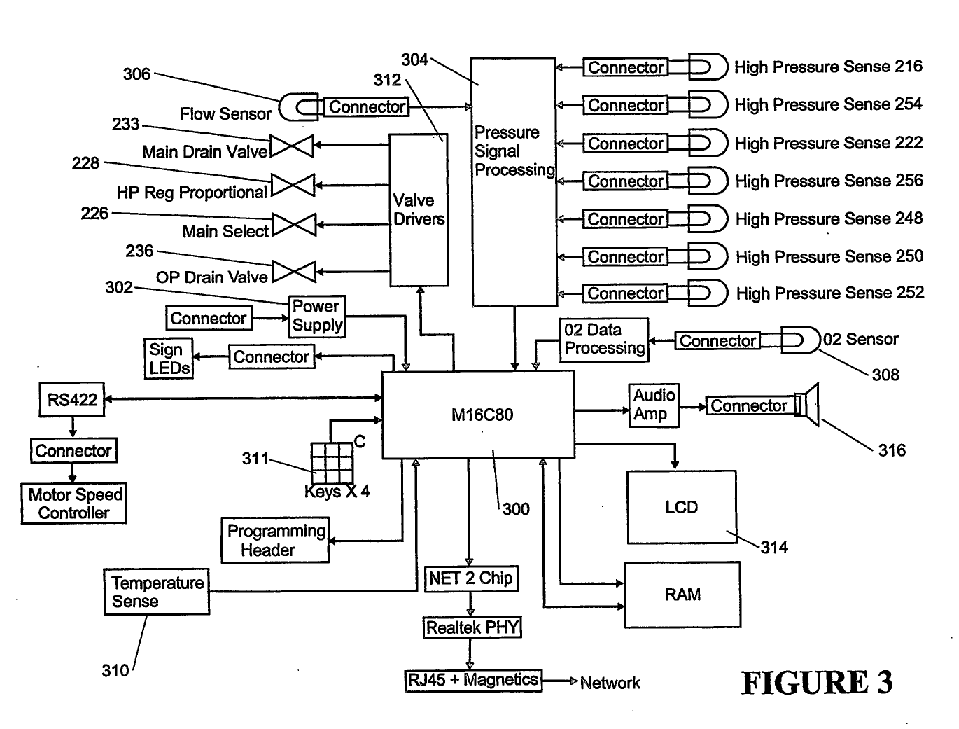

Referring to FIG. 3, one embodiment is shown with a Microcontroller 300 for controlling the gas source 100. Power Supply Unit (PSU) 302 supplies a 5V DC voltage to Microcontroller 300. The pressure and flow sensors (shown also in FIG. 2) connect to Microcontroller 300 through signal processing 304 (including an A/d with a pulse output which is supplied to a timer on the microcontroller 300). Other data signals may include oxygen flow sensor 306, temperature and/or humidity sensor 310, and keypad and/or user interface 311. Microcontroller 300 provides control signals to each valve driver 312 to control individual valves. Microcontroller 300 also controls a Liquid Crystal Display (LCD) or user interface 314 on the exterior of the gas source casing, and an audio speaker (used for warnings, power gone down and mask should be removed) 316.

The microcontroller 300 has 8 functions (not in any particular order):
1. Closed loop control of the compressor motor based on pressure in the initial receivers. This pressure is kept constant by varying the motor speed control.
2. Monitoring of various pressure points throughout the circuit for operation within set limits
3. To control the water drain valve of the filters through regular timed valve operation
4. To control the flow rate of air through the membrane to ensure optimum efficiency of the system This is done by measuring the flow rate via the sensor and closed loop control on the proportional flow valve.
5. To put a constant air load on the system by varying the over pressure valve in the system. This is required as clients put demand on the system by breathing
6. To provide constant control of the oxygen level by changing the flow and load variables of the system through the control loops for flow and load.
7. To provide a starting up period for the system where the pressure and oxygen level are stabilised and then indicating to the client system that the system can start
8. To provide a closing down period where the system receivers and lines are drained of air through the exhaust valve and the over pressure valve

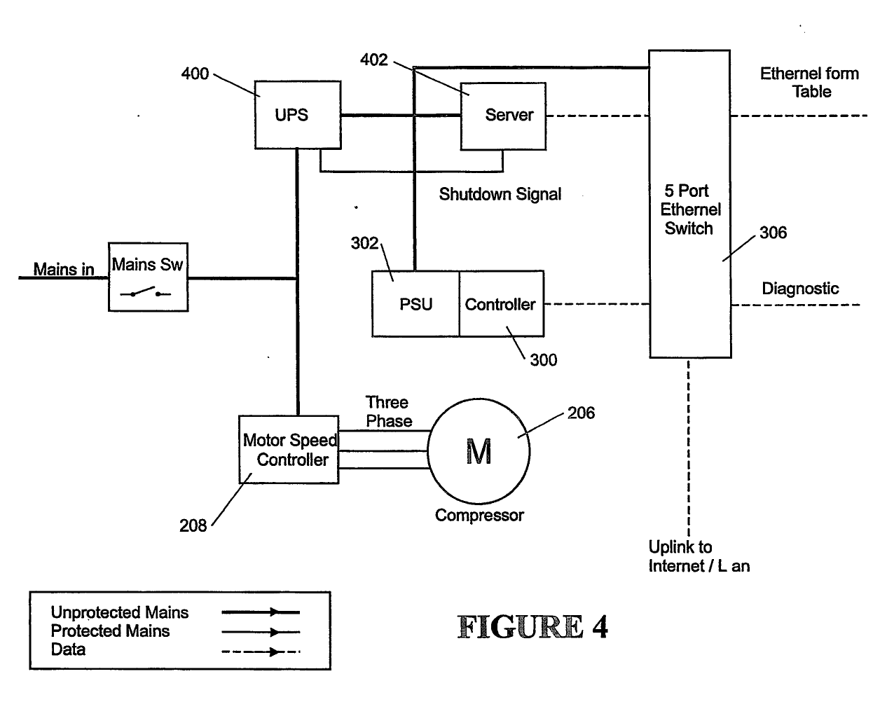

Referring to FIG. 4 in one embodiment the electrical power is supplied by a single phase AC supply, although three phase or DC could also be used. The mains supply is connected directly to the Motor Speed Controller 208 and an Uninterruptible Power Supply (UPS) 400. Motor Speed Controller 208 provides a variable 3-phase AC supply to Motor 206. UPS 400 provides a filtered and/or backup single phase AC supply to a computer server 402, Power Supply Unit (PSU) and a network switch 306, for example a 5 port Ethernet switch (hereinafter “supply switch”). PSU 302 supplies low voltage DC to the main microcontroller 300, as well as the various client node controllers. All the client nodes in the system, and the microcontroller 300 connect to the server 402 and to pick up configuration data using the supply switch 306 and TCP/IP protocol to communicate data

The server 402 is used to store all information about the system configuration, clients, and also the client programs. In addition the server logs all operational performance data of each client node and the plinth. All relevant pressures, flows, motor speeds, oxygen levels are logged for the plinth every 30 seconds and all relevant pressures, flows, oxygen levels, oximeter readings, valve positions are logged every 5 seconds for each client node. In addition to this a booking system for clients on the nodes is incorporated into the database. In addition to client data, advisor data records are also stored. Advisors are divided into technical, medical, training and administration. Each client is assigned one or more training/medical advisors. All technical changes to the system are done via a technical advisor and any operation on the system is logged against that advisor. The admin advisors control the stopping and starting of the system and also the bookings on the system. A messaging system between all advisors and clients is implemented that allows trainers/medical/admin advisors to leave messages for clients and any advisor to leave a message for any other advisor. Access to the data and the messaging/booking system is provided via an internet webserver resident on the server. This allows monitoring of the system remotely and globally),

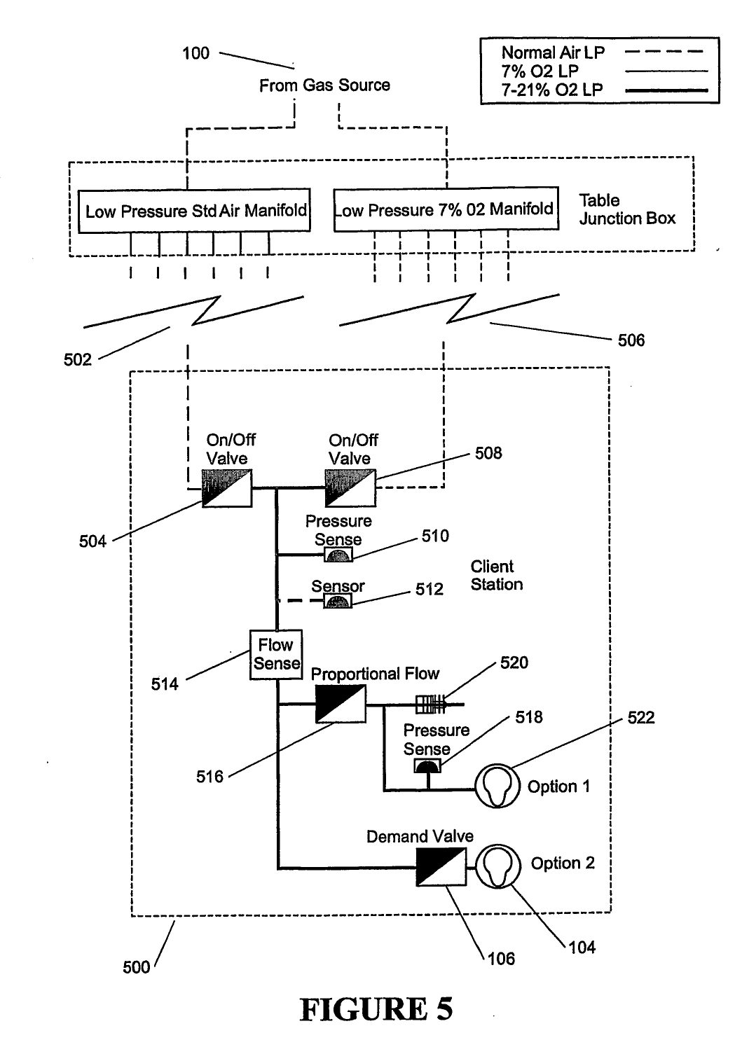

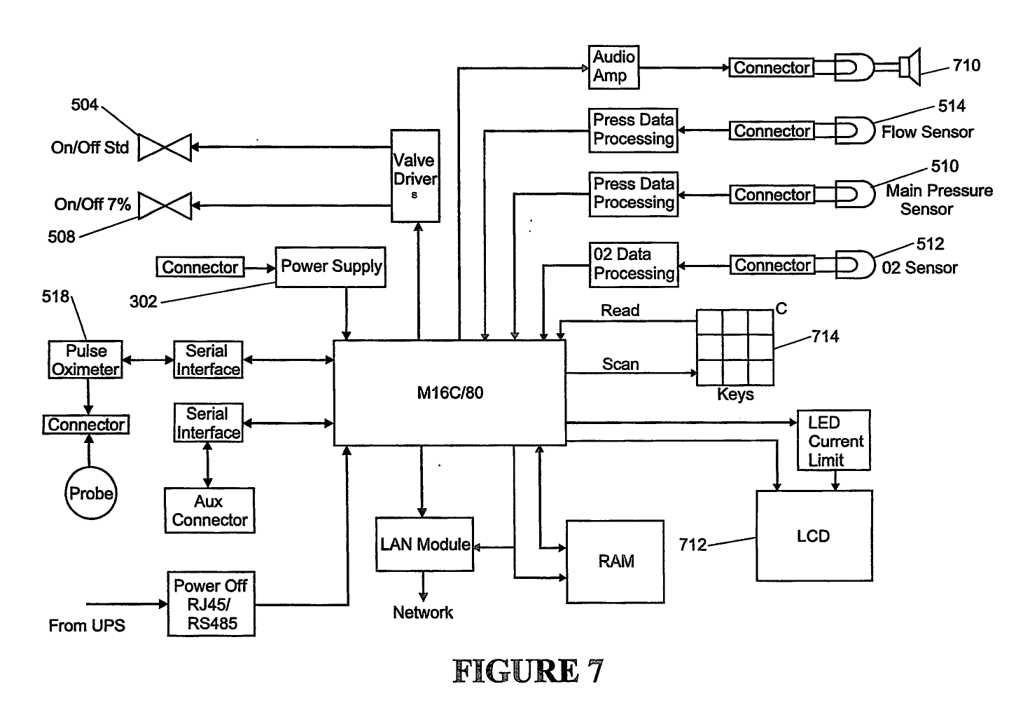

Referring to FIGS. 5 and 7, one embodiment of the client node controller 500 is shown. The gas source 100 includes a normal air conduit 502 with valve 504 and a 7% O₂ conduit 506 with valve 508. The output from the two valves is mixed and includes pressure sensing 510, O₂ level sensing 512 and flow 514 sensing. The valves are controlled to give the required O₂ level for that user. The user receives the gas from a demand valve 106 (similar to a SCUBA mouthpiece) through a mask 104. In an alternative embodiment a pressure sensor 518 is used by client node controller 500 to vary the demand flow with proportional valve 516 delivered through mask 522. This avoids the need for the more mechanically complicated demand valve previously described. Excess flow is vented through silencer 520

In one embodiment the user keeps the mouthpiece 516 in the whole time. The client node controller 500 switches the users air from a fixed period on normal air and then a fixed period on hypoxic air.

Pulse oximetry provides estimates of arterial oxyhemoglobin saturation (SaO2) by utilizing selected wavelengths of light to non-invasively determine the saturation of oxyhemoglobin (SpO2). The pulse oximeter 518 is used to control the oxygen level in the clients' blood. As the SpO2 level drops through the upper threshold client node controller 500 switches the oxygen mix from hypoxic to normoxic. The SpO2 level will continue to fall (lag in the cardiovascular system) and then it will pass through the lower threshold and then plateau and start to rise again, as the level rises through the lower threshold again the client node controller 500 switches to hypoxic air again. The SpO2 level will increase through the upper threshold, plateau and then pass back down through the upper threshold again and the control cycle will continue. The client remains on this hypoxic controlled air flow for a fixed period (say 5 minutes) and then reverts back to normoxic for a similar fixed period, over which time the oxygen level will rise back to normal.

Along with the oximeter SpO2 level, the heart rate is recorded, and by monitoring the pressure in the demand valve line the number of breaths per minute are also logged. If at any time the number of breaths per minute exceeds a set level or the heart rate rises above a set level or the SpO2 level falls below a set level then an alarm is sounded on the audio interface, an indication is shown on the user screen and a warning message is sent to the user administrator. In addition the client node switches to normal air and the user is prompted to remove their mask.

While in one embodiment a demand valve is used to supply the user, alternatively an electronic demand valve (which will monitor the pressure in the mask and then control the delivery valve directly). This will allow a standard mask to be used and also remove the high cost of the mechanical type demand valves.

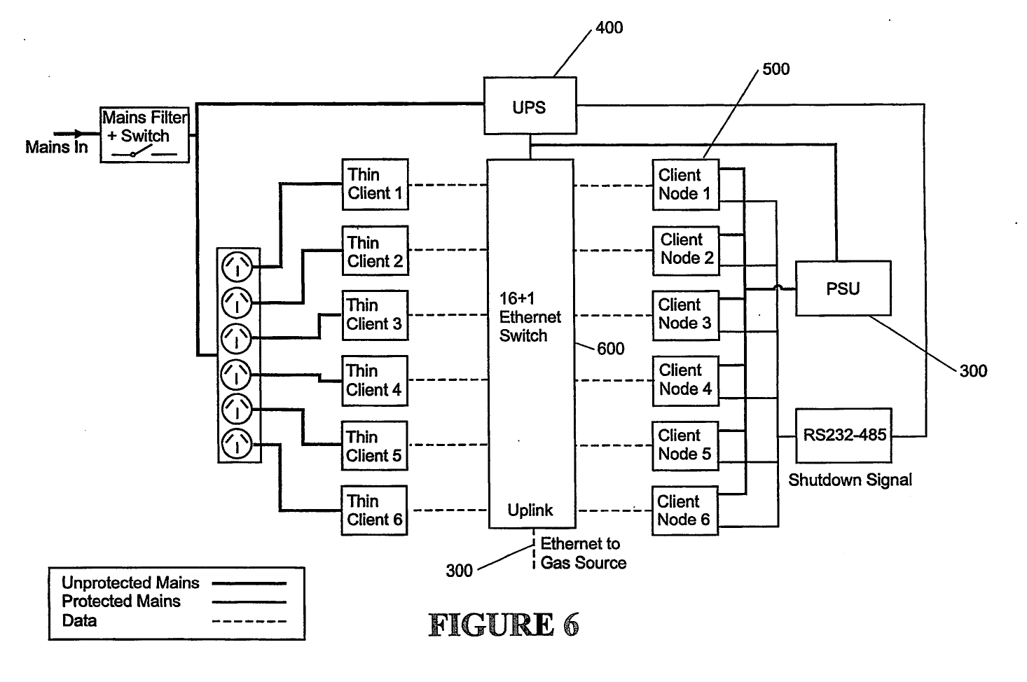

Referring to FIG. 6, one embodiment of each client node controller 500 is connected to a network switch 600, for example a 16 port Ethernet switch (hereinafter “client switch”). The client switch 600 is connected to the supply switch 306 to allow data exchange. Each client node controller 500 receives a low voltage DC supply from the PSU 302.

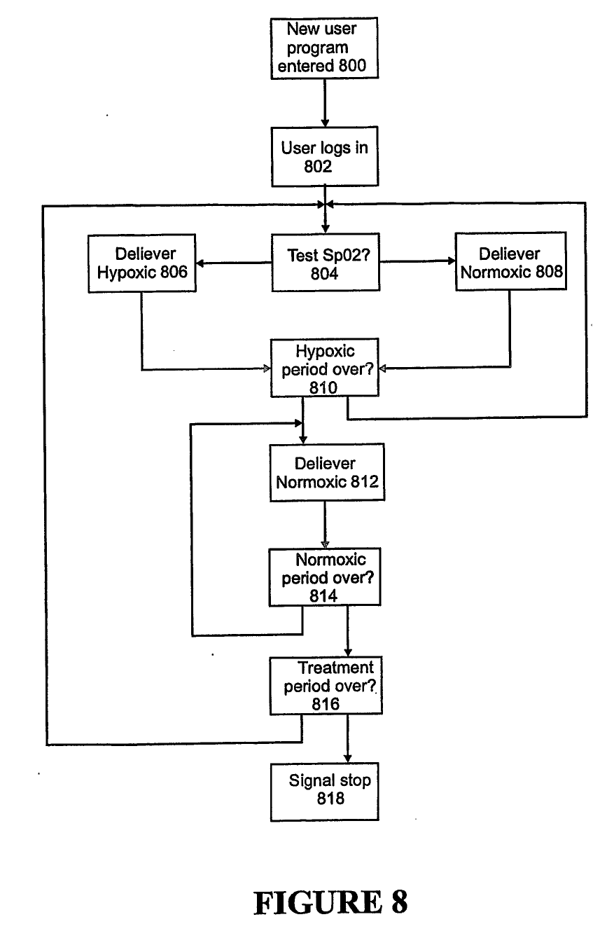

Referring to FIG. 8 one embodiment of the operating procedure is shown. The client is screened for any medical issues before starting the training programme and a blood test is carried out to ensure there is a sufficient level of iron. Iron storage is necessary for the physiological adaptation of enhancing the number of red blood cells. In the case of low iron levels supplementation is recommended. Unlike other forms of altitude exposure a strict protocol is followed to allow for adaptation to the lack of oxygen (altitude air) and to ensure adequate safety standards are maintained.

The altitude simulator extracts oxygen from the air to achieve oxygen levels as low as 9% (equivalent to 22,000 feet altitude) which is less than half the oxygen content at sea level (20.9%). The client breathes this through an oral and/or nasal mask.

The blood oxygen content may be continuously monitored using an oxygen monitoring device called a Pulse Oximeter that is clipped to the client's ear. This ensures the desired mix of normoxic air (room air) and hypoxic air is delivered in respect to the level of the client's blood oxygen de-saturation.

The training programs may be preset in the system (stored on the server) These are set up by qualified personnel. A client is assigned to a particular program by the training or medical advisor assigned to that client. That program varies each day and each client node can be running a different program. The client will make a booking on the system and then when the client arrives they sign in at the administration desk. The client is then told which client unit they will be using and the correct data for that day in the client program along with all the client messages and configuration data is sent from the server to the assigned client unit.

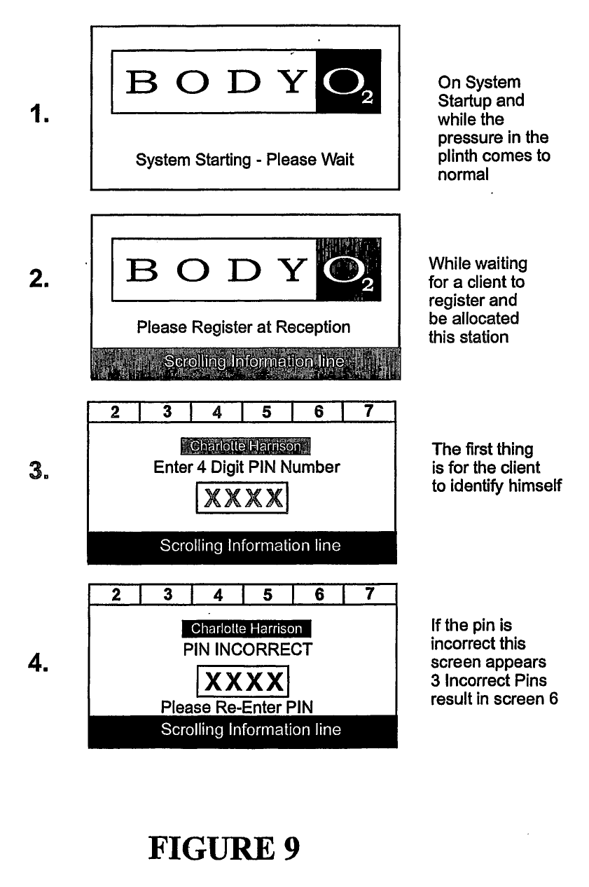

Upon receipt of the client data the unit then displays the client name and then prompts for the client to enter his pin number on the numeric keypad as seen in FIG. 9. If the pin is entered correctly then the client is prompted to put on the mask and press the- start button. The client can stop or pause the program at any stage through the program. The client screen shows current relevant data for the session and a variety of screen based reports are available on the LCD throughout the session. At the end of the session, graphs of the session data can be printed on the system printer or retrieved later via the web interface.

An example programme is made up of five, one hour per day sessions, although the clients' initial programme might be 15 days. Each once hour session might consist of repeated cycles of 5 minutes normoxic air exposure and 5 minutes hypoxic exposure breathing through the mask this process. Throughout the five-day programme, the body progressively adapts to the hypoxic exposure. The oxygen content in the inspired air is gradually reduced to ensure that an optimal hypoxic stimulus is maintained, maximising the physiological adaptation.

When using SAT, the negative effects of chronic altitude exposure are avoided due to the intermittent hypoxic exposure and the absolute control over the level of oxygen de-saturation.

Entertainment Application

Clients can access and surf the net while receiving their SAT programme via the computer screen in front of the client. They may also access a video from the gym/sports club/place at which the equipment is at. The video/DVD may be a Fitness Promotional video, or a video of a recent game so the players can watch how they performed.

Education or Instruction

The computer screen may also be a video of what simulated altitude training is all about, and how to use the equipment correctly.

Advertising

Advertising content may be displayed interactively through the thin client.

Parameter Monitoring/Display

Part of the equipment is a pulse oximeter which is clipped to the clients ear. This reads the clients heart rate and blood saturation level. It is the blood saturation that the simulator determines what the correct level of hypoxic air mix is delivered to the client. These vital signs are shown on the screen so the client can see how they are responding to different air mixes being delivered.

Self Diagnostics

When the system is turned on, it automatically performs a self diagnostic test to ensure all parts are working as they should. There are a number of electronic readers and meters placed within the simulator that each measures particular parts of the unit. If there is a fault or one reading is not what it should be, a message is automatically forwarded to our service contractors who know (via remote) that the simulator is not working and which part is at fault.

It will be appreciated that any of these media can be varied in relation to any of the data or variables available to the controller. For example, different media could be displayed when the user is on hypoxic compared to normoxic air.

Applications

Almost anyone can benefit from altitude exposure. It will improve the general health, fitness, performance, endurance, recovery and rehabilitation of elite and recreational athletes.

The training course is suitable for:
human—sport and athletic performance enhancement
horse—performance enhancement, speed, endurance, heart recovery, muscle recovery
fertility—increasing the fertility rate (being able to conceive more easily):human, horse, chicken, camel, all animals
camel—performance enhancement
military—being able to acclimatise to higher altitudes, increasing level of fitness, performance enhancement
health—animals and humans, reduces cholesterol, speeds up metabolism
weight loss—humans
all high altitude situations
aviation—training for pilots
asthma treatment or weight loss

The results for all participants are improved peak performance, endurance and recovery.

The typical response to this form of altitude exposure which occurs progressively through the course is as follows:
Increased energy levels and a significant increase in oxygen uptake
Reduction in resting and exercising heart rate
Decreased blood pressure
An improved immune function
Burns up fatty acids thereby causing weight reduction

A reduction in lactate build up as a result of increased buffering capabilities; this means the production and clearance of waste product and toxins is improved, causing less actual muscle fatigue, enabling the athlete to perform better
An increase in red blood cell production assisting with increasing the oxygen binding capabilities and therefore the amount of oxygen carried in the blood increases
Up to a 20% reduction in blood cholesterol levels

The net effect of these and many other changes that occur with this form of altitude exposure is a marked improvement in the oxygen extraction, delivery and utilisation. Put simply, this means increased amounts of oxygen is available to be delivered and utilised by the body with greater levels of efficiency.

The typical response to SAT is an improvement in athletic performance. More specifically, an improvement in:
Speed over both short and long distances
Improved endurance and stamina
An increased ability to cover a greater distance before needing to rest
Increased power due to increased energy levels
Reduced recovery time between intense activities
Increased ability to adapt to high altitude environments
Greater control of breathing during exercise especially in intense activities requiring near maximum or maximum performance

The sports applications of this form of training include;
Acceleration and improvement in pre-season fitness training
Improvement of recovery from intense training sessions.
Recovery between intervals of high intensity effort
Improves peak performance levels
Improved ability to maintain high levels of fitness throughout a competition period
Assistance in the maintenance of fitness in injured or ailing athletes forced to reduce their training intensity
Assistance in the final preparation for major sports event, including those held at altitude
Assistance in final preparations in acclimatising prior to travelling to altitude, shortening the length of time initially required at altitude before performance levels can be increased
Improved anaerobic and aerobic conditioning—maximising specific training goals
Ability to maximise training time when incorporated into the correct sub-phases of training

The benefits of this training regime are equally applicable to individuals in terms of:
Increased general fitness
Corporate health benefits through reduced stress levels and improved concentration and mental awareness
Asthma management, reducing wheeze and medication requirements, giving the feeling of a greater easing of breathing in the chest
Chronic fatigue relief
   Alternative Treatment Systems

The system described above is suitable for delivery to most types of animals. One skilled in the art would appreciate slight variations will be necessary to allow the present invention to be used on plants or micro-organisms. As an example in case of horses the only real variation required is more capacity per client. An individual horse may require up to 120 l/min. This in turn requires the receivers (1×200 l) motor (3.7 kw 3 phase) and compressor (420 l/min) to be increased. Paralleling of multiple gas sources is also possible to increase capacity.

Alternative O₂ Level Regulation

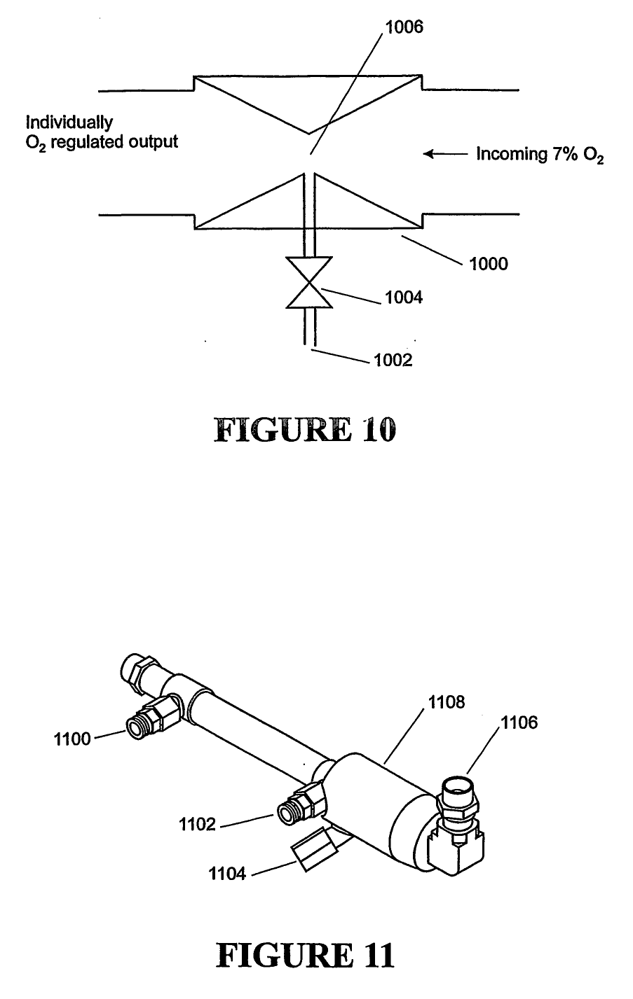

Referring to FIG. 10 one embodiment is depicted using a venture 1000 to regulate the level of oxygen.

The capacity of the simulator is able to be increased. Depending on the type of venturi used and the % of oxygen that the simulator can produce and the required oxygen levels for the programme, a venturi can be sized to increase the flow by up to 3 times. This increases the number of people that the simulator can service.

In individual mix simulators the venturi is used to do the final mixing of the air to achieve the desired oxygen levels. The intake of atmospheric air 1002 into the venturi 1000 is restricted by a proportional valve 1004 that is set depending on the required oxygen levels. The atmospheric air is drawn into the throat 1006 and mixes with the higher pressure depleted oxygen gas depending on the flow therethrough. This air is then stored in a flexible storage bag, that is connected to a valve that sends the air to either A or B station.

When used in an Equine application the use of a venturi for the final mixing has the additional ability of scenting the air. Chopped up apples or carrots can be placed in the bottom of the storage cabinet. As the venturi is in this cabinet, and the final mixing is done their scent is introduced, this helps the horse to accept the mask being put on, and the forced flow of the air in the mask.

Membrane Temperature Regulation

Membrane performance changes as the temperature rises. Due to the after cooler being installed pre membrane; (to reduce the moisture in the system) it can take a considerable time for the temperature to rise up to a level that the membrane works efficiently and consistently. In fact in cooler conditions the membrane may never reach its efficient operating temperature.

By utilising an after cooler to reduce the moisture in the system, then a re-heat function achieved by the heat exchanger before the compressed air enters the Membrane the possibility of liquid moisture passing through the Membrane is significantly reduced, improving the life of the Membrane.

The heat exchanger utilises the heat from the compressor, this means that there is no extra power consumption required.

The Heat exchanger is an air to air Heat exchanger, consisting of a helical copper middle section 1108 where the hot air from the compressor 1106 is passed through the middle to the after cooler 1100. Outside of this is an outer cooper tube where the compressed air (after the after cooler, receiver and air filter) 1102 is then passed through, passing over the hot middlehelical section for a tuned length before leaving the heat exchanger 1104.

The air to air heat exchanger with the utilisation of a temperature controller, and a three position, centre open, five port Temperature control valve completes the temperature control system of the compressed air to the membrane.

The temperature control system gives improved Membrane life and consistent Membrane performance keeping the oxygen depletion consistent. The more stable the system is pre electronic control of the mixing, the more accurately and quickly the required mixing levels are achieved.

On the bottom of the heat exchanger is an evaporating chamber, where the condensate from the air receiver and filter is forced in under pressure at 5-10 minute intervals by the drain valve. The moisture is evaporated away in this chamber and exhausted into the compressor cooling air exhaust. This means that the manual draining of the system/container is not required.

In warm up state all of the air is passed through the Heat exchanger to bring the Membrane up to ideal operating conditions (between 40-45° C.) quickly (approximately 5 minutes at ambient temperatures of 20° C.).

In operating mode 50% of the air is passed through the Heat exchanger and 50% of the air bypasses the Heat exchanger, and is remixed.

In over temperature mode all of the air bypasses the Membrane in order to keep the temperature under 45° C., protecting the Membrane from getting too hot, increasing the life of the Membrane.

The Temperature controller controls the Temperature control valve in-between these three states, trying to maintain between the membrane to 40-45° C.

### Alternative Embodiments

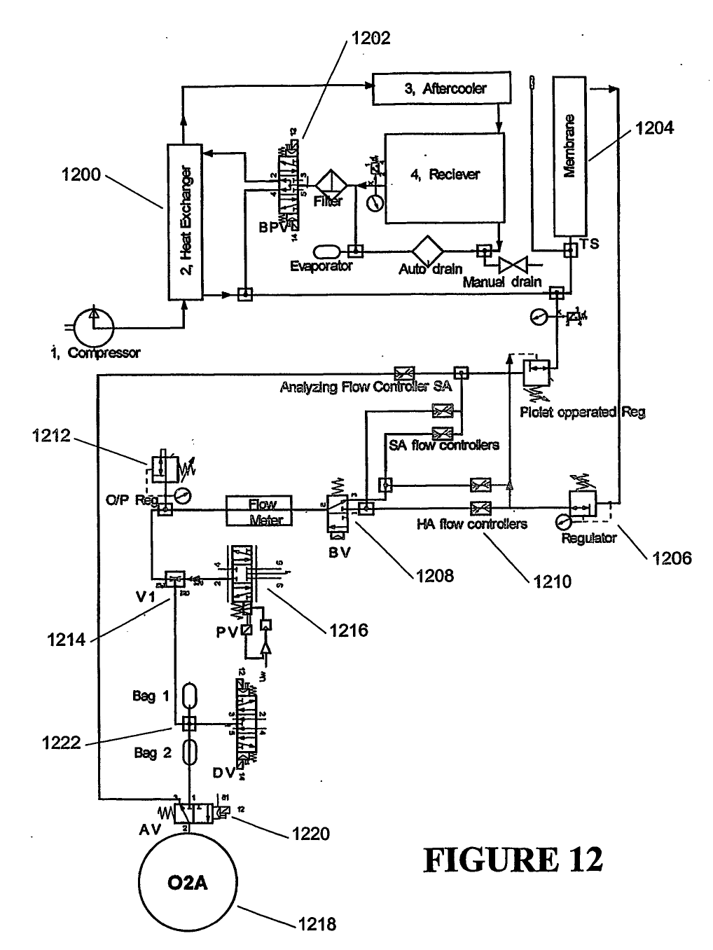

Referring to FIG. 12 a pneumatic diagram showing an alternative embodiment incorporates the heat exchanger and venturi previously described. In this case a heat exchanger 1200 is connected with proportional valve 1202 to obtained various levels of heating with respect to the heat entering the membrane 1204. In one example proportional valve 1202 is selectable in three states to achieve either maximum heating, an operating heating setting and no heating. The compressor outputs air at around for example 6 bar whereas the oxygen depleted gas output from the membrane is regulated by regulator 1206 to 5 bar. The regulated depleted oxygen; and normal oxygen level gases are then mixed through proportional valve 1208 to give a predetermined level of depleted oxygen at around 3.5 bar for example. It is possible in FIG. 12 to provide more than one predetermined level of depleted oxygen for example flow controllers 1210 may be hand calibrated to give for example a first setting of 7% oxygen, and a second setting of 11.5% oxygen for applications requiring variation. Over pressure regulator 1212 controls the 7% oxygen gas to constant pressure and flow for example 3.5 bar. Venturi 1214 then regulates the 7% oxygen gas to whatever proportion oxygen the system has predetermined for the current user at the current time. This is achieved by proportional valve 1216 adjusting the ambient air which is introduced to the throat of the venture. Oxygen sensor 1218 may be selectively connected to the outlet gas or the normal oxygen level gas. Particular stations through which the user may be connected are supplied by the output through selectable valve 1220. The output flow and pressure is further buffered by two bags 1222.

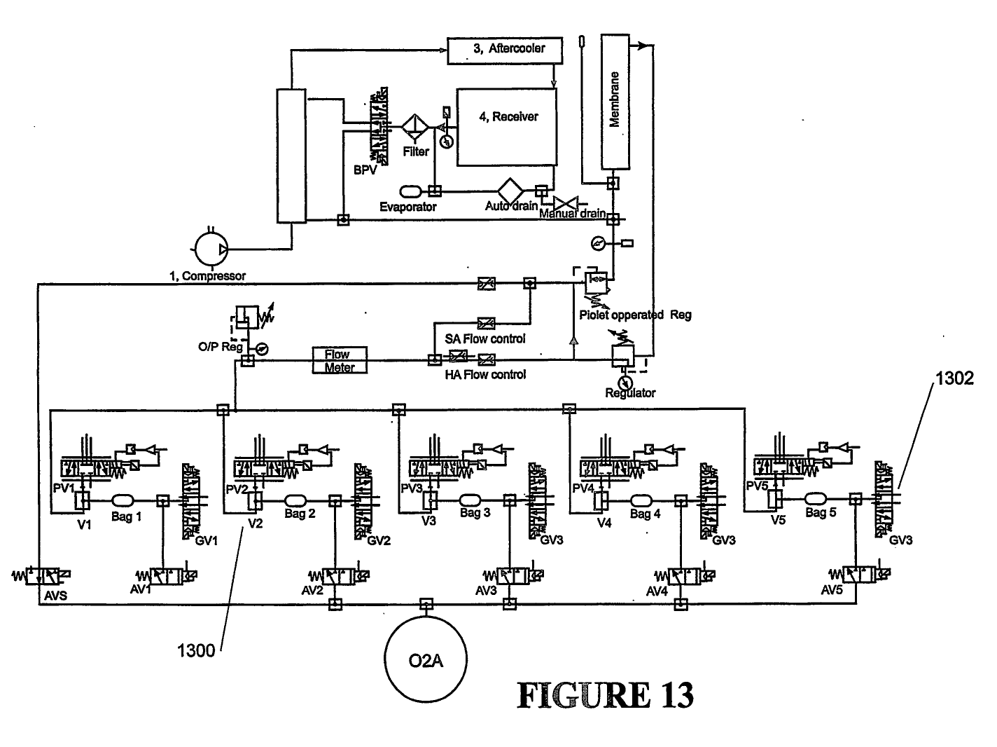

Referring to FIG. 3 the predetermined depleted oxygen level combining may be simplified where only one predetermined level of oxygen is required. As is shown in FIG. 13 individual venturi's 1300 and demand valves 1302 can be supplied for each user station or alternatively a lesser number could be switched between each one, for example users could alternate between rest periods and oxygen depletion periods.

The system could also contemplate for example a less aggressive oxygen depletion if pulse oximetry data was not provided, and if it was available to provide a more aggressive strategy as and when the blood oxygen level of the user allowed.

1-30. (canceled)

31. A system for modifying the composition of air and delivering said modified gas to a recipient comprising:
at least one source of gas configured to vary said composition and deliver said modified gas to said recipient depending on a control signal;
at least one sensor configured to determine the oxygen level of said modified gas; and
at least one controller configured to receive an output of said sensor and provide said control signal to said source based on said sensor output, a predetermined desired level or range and at least one historical value of said sensor output.

32. A system for modifying the composition of air and delivering said modified gas to at least one recipient comprising:
at least one source of gas configured to vary said composition and deliver said modified gas to each said recipient depending on a control signal;
at least one controller configured to receive identification of each said recipient and provide said control signal to said source based on the progress through a predetermined oxygen profile stored in relation to said recipient.

33. The system as claimed in claim 31 including a non-invasive sensor configured to determine the level of oxygen in the blood of said recipient and said controller is configured to provide a control signal based, at least in part, on the output of said blood oxygen level sensor.

34. The system as claimed in claim 31 wherein said system is configured to independently deliver gas to two or more recipients at the same time.

35. The system as claimed in claim 31 wherein said source further comprises at least two inputs; a first input configured to receive normoxic (normal oxygen level) gas or ambient air, and a second input configured to receive hypoxic (oxygen reduced, depleted or bereft) gas.

36. The system as claimed in claim 35 wherein said source further comprises at least one mixer configured to vary the proportion of gas from said first input and said second input according to said control signal.

37. The system as claimed in claim 36 wherein said source further comprises at least two said mixers, at one mixer configured to supply each said recipient.

38. The system as claimed in claim 31 wherein said controller is configured to receive the heart rate of said recipient and provide said control signal to said source also based on said heart rate.

39. The system as claimed in claim 31 said controller configured to receive the breathing rate of said recipient and provide said control signal to said source also based on said breathing rate.

40. A system for modifying the composition of air and delivering said modified gas to a recipient comprising:
means for varying said composition and delivering said modified gas to said recipient depending on a control signal;
means for determining the oxygen level of said composition; and
means for providing said control signal to said source based on said determined oxygen level, a predetermined desired level or range and at least one historical value of said determined oxygen level.

41. A system for modifying the composition of air and delivering said modified gas to at least one recipient comprising:
means for varying said composition and delivering said modified gas to each said recipient depending on a control signal;
means for receiving identification of each said recipient and provide said control signal based on progress through a predetermined oxygen profile stored in relation to said recipient.

42. A method for modifying the composition of air and delivering said modified gas to a recipient comprising:
varying said composition and delivering said modified gas to said recipient depending on a control signal;
determining of the oxygen level of said composition; and
providing said control signal to said source based on said determined oxygen level, a predetermined desired level or range corresponding to said composition and at least one historical value of said determined oxygen level.

43. A method for modifying the composition of air and delivering said modified gas to at least one recipient comprising:
varying said composition and delivering said modified gas to each said recipient depending on a control signal;
receiving identification of each said recipient; and
providing said control signal to said source based the progress through a predetermined oxygen profile stored in relation to said recipient.

44. A system for modifying the composition of air and delivering said modified gas to at least one recipient comprising:
at least one oxygen reducer reducing the oxygen component of said gas;
at least one heater adapted to heat said gas before it enters said reducer and/or adapted to heat said reducer directly.

45. The system as claimed in claim 44 wherein said heater is configured to provide a first level of heating at start up and a second operating level of heating.

46. The system as claimed in claim 45 wherein said heater is configured to maintain at least a component of said reducer within a predetermined range.

47. The system as claimed in claim 46 wherein said range is 40-45° C.

48. A system for modifying the composition of air and delivering said modified gas to at least one recipient comprising:
at least one source of low oxygen gas at above ambient pressure;
at least one source of ambient air;
at least one mixer or venturi including a gas inlet configured to connect to said low oxygen source, a variable throat inlet configured to connect to said ambient air source, and at least one controller configured to vary said throat inlet to achieve a predetermined proportion or range of oxygen at the gas output from said mixer.

49. The system as claimed in claim 48 further comprising an oxygen sensor providing an indication of the oxygen level at said gas outlet, and said controller configured to vary said throat inlet at least based on said predetermined proportion and said indication of said oxygen level.

50. The system as claimed in claim 48 further comprising a receptacle to receive scented material proximate or integrated with said throat inlet.

51. The system as claimed in claim 35 wherein said controller is configured to periodically cause said source to supply substantially normoxic gas for a period of time between periods of time supplying modified gas.

52. The system as claimed in claim 32 including a non-invasive sensor configured to determine the level of oxygen in the blood of said recipient and said controller is configured to provide a control signal based, at least in part, on the output of said blood oxygen level sensor.

53. The system as claimed in claim 32 wherein said system is configured to independently deliver gas to two or more recipients at the same time.

54. The system as claimed in claim 32 wherein said source further comprises at least two inputs; a first input configured to receive normoxic (normal oxygen level) gas or ambient air, and a second input configured to receive hypoxic (oxygen reduced, depleted or bereft) gas.

55. The system as claimed in claim 54 wherein said source further comprises at least one mixer configured to vary the proportion of gas from said first input and said second input according to said control signal.

56. The system as claimed in claim 55 wherein said source further comprises at least two said mixers, at one mixer configured to supply each said recipient.

57. The system as claimed in claim 54 wherein said controller is configured to periodically cause said source to supply substantially normoxic gas for a period of time between periods of time supplying modified gas.

58. The system as claimed in claim 57 wherein said controller is configured to receive the heart rate of said recipient and provide said control signal to said source also based on said heart rate.

59. The system as claimed in claim 57 said controller configured to receive the breathing rate of said recipient and provide said control signal to said source also based on said breathing rate.
